(19) United States
(12) Patent Application Publication
(10) Pub. No.: US 2021/0188762 A1
(43) Pub. Date: Jun. 24, 2021

(54) PROCESS FOR PRODUCING PHENALKAMINES

(71) Applicant: Evonik Operations GmbH, Essen (DE)

(72) Inventors: Gauri Sankar Lal, Whitehall, PA (US); Michael Cook, Macungie, PA (US); Emmanouil Roumpelakis, Breinigsville, PA (US); Gamini Ananda Vedage, Bethlehem, PA (US)

(21) Appl. No.: 17/045,796

(22) PCT Filed: Apr. 9, 2019
(86) PCT No.: PCT/EP2019/058864

§ 371 Date: Oct. 7, 2020

Related U.S. Application Data
(60) Provisional application No. 62/655,282, filed on Apr. 10, 2018.

(51) Int. Cl. C07C 213/02; C08G 59/50

(52) U.S. Cl. CPC C07C 213/02; C07C 215/50; C08G 59/504

(57) ABSTRACT

The present invention relates to a new method of making phenalkamines, products produced by such method, and use of such products. The method provides for phenalkamines obtained by an amine exchange reaction of a cardanol derived Mannich base with a compound with at least one alkylene or aralkylene group and at least two amino groups. These products may be used to cure, harden, and/or crosslink an epoxy resin. The curing agent compositions of this invention are of low viscosity and can be used neat or dissolved in a minimum amount of an organic solvent or diluent to effect cure of epoxy resins.

### BACKGROUND OF THE INVENTION

The Mannich reaction is based on the reaction of an aldehyde, generally formaldehyde, a phenolic compound and an amine. Various forms of phenolic compounds, amines and aldehydes have been utilized in this reaction. The Mannich base products are particularly suitable for curing epoxy resins.

Phenalkamine curing agents are a class of Mannich bases obtained by reacting cardanol—an extract of cashew nutshell liquid, an aldehyde compound, such as formaldehyde, and an amine. Generally, they are produced from the reaction of one molar equivalent of cardanol (structure according to formula (I) below) with one to two molar equivalent of an aliphatic polyethylene polyamine and one to two molar equivalent of formaldehyde at 80-100° C., Sometimes aromatic polyamines have also been used for this reaction. The commercially available phenalkamines based on ethylenediamine and diethylenetriamine as the amine sources are available from multiple industry suppliers, e.g. NC 541 and NC 540 available from Cardolite Inc, and Sunmide CX-105 and CX-101 from Evonik Corp.

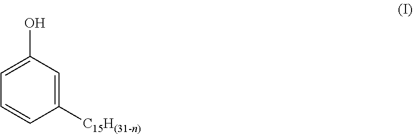

Phenalkamines are good epoxy resin hardeners for room temperature or low temperature curing applications. In formulated systems with liquid epoxy resins, resultant coatings exhibit excellent barrier properties and as a result they are one of the key curing agent technologies used in the marine and heavy duty protective coating markets. More recently, the technologies have found further uses in civil engineering and structural adhesive applications.

GB Patent No. 1,529,740 describes phenalkamines as mixtures of poly(aminoalkylene) substituted phenols (structure according to formula (II) below) prepared from cardanol with polyethylene polyamines and formaldehyde. In general, it is not possible to easily control the molecular weight distribution of these products and hence they are usually highly viscous liquids.

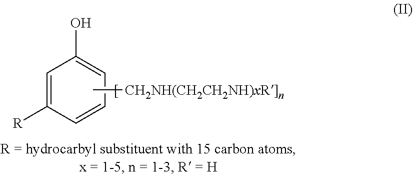

U.S. Pat. No. 6,262,148 B1 describes compositions of phenalkamines bearing aromatic or alicyclic rings. These compositions were prepared from cardanol with aldehydes and alicyclic or aromatic polyamines. International Application Publication No. WO 2009/080209 Al describes the preparation of epoxy curing agents comprising phenalkamines blended with polyamine salts. These curing agents were used to enhance the rate of cure of epoxy resins.

There is a need in the art for phenalkamine curing agents for epoxy resins which can accelerate the cure speed at sub-ambient temperature (e.g. 5° C.) and which can be used with minimal amount of volatile organic solvents. Consequently, liquid phenalkamines of low viscosity are highly desirable.

### BRIEF SUMMARY OF THE INVENTION

The present disclosure provides a new method of making phenalkamines, products produced by such method, and use of such products. These phenalkamines and products may be used to cure, harden, and/or crosslink an epoxy resin. This invention solves problems associated with phenalkamine curing agents by providing compositions which are of low viscosity (<3000 mPa·s at 25° C.) which can be used neat or dissolved in a minimum amount (<20 wt%) of an organic solvent or diluent to effect cure of epoxy resins. In addition, these phenalkamine curing agents can provide dry cure of epoxy coatings at ambient temperature (25° C.) in <8h or at 5° C. in <16 h. This invention relates to the new method of producing phenalkamines represented by the structures in formulas (III), (IV), (V) and (VI) below.

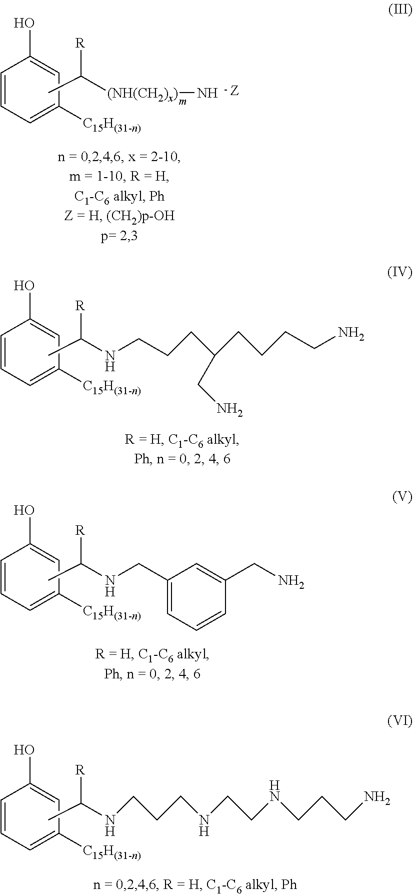

Phenalkamines of the structure according to formula (III) cannot be obtained cleanly by the traditional Mannich reaction process since several competing reactions take place to give a complex mixture of products with the amino substituent at both the ortho and para positions to the hydroxyl substituent of cardanol. In addition, there is a rapid cyclization reaction between the 1,2 diamino groups or 1,3-diamino groups of the amines with aldehydes which reduces the overall —NH content of the product. The scheme below outlines the cyclization process of the 1,2-diamino group and 1,3-diamino group with formaldehyde.

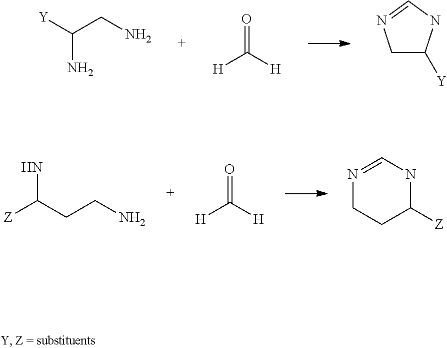

The present invention provides for a method of producing this class of phenalkamines obtained by an amine exchange reaction of a cardanol derived Mannich base (structure according to formula (VII) below) with a compound with at least one alkylene or aralkylene group and at least two amino groups (structure according to formulas (VIII), (IX) and (X) below), where the compound can comprise at least two or more alkylene or aralkylene groups, and where linear alkylene or aralkylene groups are preferred.

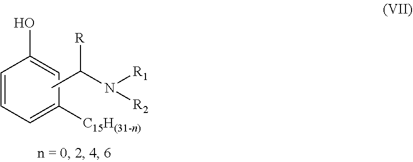

R═H, C₁-C₆ alkyl, or phenyl, R₁, R₂=alkyl or aryl substituent of the secondary amine

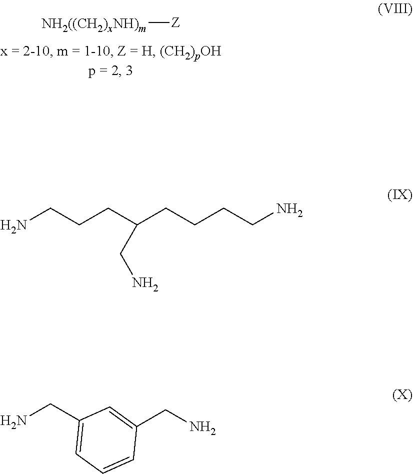

The cardanol derived Mannich base is the product obtained by reacting cardanol with a secondary amine (R₁R₂NH) and an aldehyde (RCOH). The secondary amine is represented by the structure below:

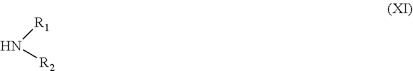

with R₁ and R₂ being, independently of each other, a C₁-C₆ alkyl or aryl group.

The compound with at least one alkylene or aralkylene group and at least two amino groups can have at least one ethylene group, at least one propylene group, at least one butylene group, at least one pentylene group, at least one hexylene group, at least one heptylene group, at least one octylene group, at least one nonylene group, at least one decylene group, at least one alkylene group with a hydroxyalkyl group and/or combinations thereof.

Preferably, curing agent compositions of the present disclosure have an amine hydrogen equivalent weight (AHEW) based on 100% solids from about 30 to about 500.

The present disclosure, in another aspect, provides amine-epoxy compositions and the cured products produced therefrom. For example, an amine-epoxy composition, in accordance with the present disclosure, comprises a curing agent composition containing the novel phenalkamine composition comprising at least one cardanol group and having at least two active amine hydrogen atoms and epoxy composition comprising at least one multifunctional epoxy resin.

The present disclosure also provides for a product produced by the method of making phenalkamines represented by the structures in formulas (III), (IV), (V) and (VI). The present disclosure further provides for the use of these products for the preparation of hardened articles and for the hardening of epoxy resins.

Articles of manufacture produced from amine-epoxy compositions disclosed herein include, but are not limited to, adhesives, coatings, primers, sealants, curing compounds, construction products, flooring products, and composite products. Further, such coatings, primers, sealants, or curing compounds may be applied to metal or cementitious substrates.

The mix of curing agent and epoxy resin often requires no induction time for obtaining contact products with high gloss and clarity. Induction time or ripening time or incubation time is defined as the time between mixing epoxy resin with amine and applying the product onto the target substrate. It could also be defined as the time required for the mix to become clear. Furthermore, the phenalkamine compositions of the present invention also provide faster amine-epoxy reaction rate, and relatively low viscosity. These unique properties provide the advantages of lower tendency to carbamate, shorter time for coatings to dry, and reduced or eliminated amount of solvent needed, the latter being an important industry requirement as coating formulators develop lower VOC (volatile organic content) containing coating systems to meet emerging environmental drivers.

### DETAILED DESCRIPTION OF INVENTION

The method for producing phenalkamines of the present invention are prepared by a two-step process. The first step involves the preparation of a Mannich base intermediate (structure according to formula (VII) below) by reacting cardanol with a secondary amine (NHR¹R²) and an aldehyde.

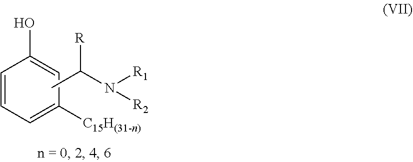

R═H, C₁-C₆ alkyl, phenyl, R₁, R₂=alkyl or aryl substituent of the secondary amine.

This intermediate is then reacted with a compound with at least one alkylene or aralkylene group and at least two amino groups in a second step to generate the phenalkamine curing agents of this invention represented by formulas (III), (IV), (V) and (VI) below.

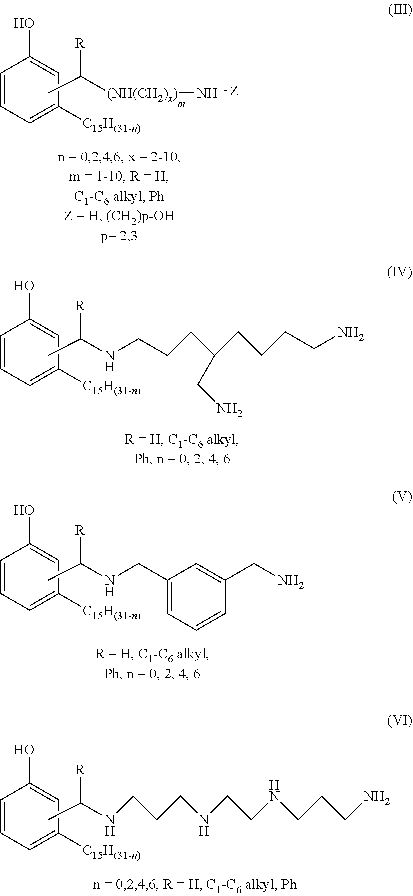

Preferably, the compound with at least one alkylene or aralkylene group and at least two amino groups, where the compound can comprise at least two or more alkylene or aralkylene groups, and where linear alkylene or aralkylene groups are preferred, are represented by the structures:

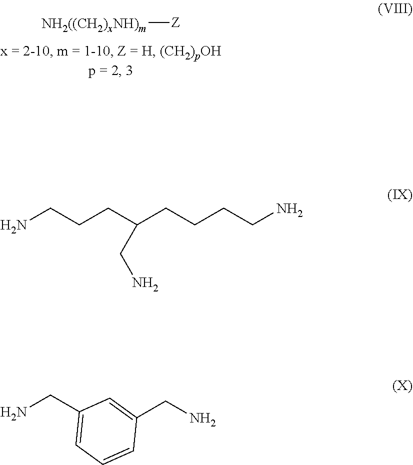

The secondary amine used to prepare the Mannich base intermediate preferably has a boiling pt. of <50° C. than the compound with at least one alkylene or aralkylene group and at least two amino groups used for the amine exchange reaction in the second step for efficient production of the phenalkamine curing agents of this invention. The secondary amine is represented by the structure below:

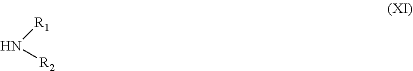

with R₁ and R₂ being, independently of each other, a C₁-C₆ alkyl or aryl group. Preferable examples of secondary amines which can be used for this process include dimethylamine, diethylamine, dipropylamine, dibutylamine, dipentylamine, dihexylamine, dicyclohexylamine, and N-methylaniline. Preferable examples of aldehydes used for preparing the Mannich base intermediate of step 1 include formaldehyde, acetaldehyde, propionaldehyde, butyraldehyde, pentanal, hexanal, and benzaldehyde.

The process to prepare the Mannich base intermediate of step 1 requires the addition of the aldehyde to a mixture of the secondary amine and cardanol at the reaction temperature. Alternately, the amine can be added to a mixture of cardanol and aldehyde at the reaction temperature. Other sequences of combining these raw materials are also possible. The reaction can be conducted in water or in an organic solvent. Suitable solvents include aromatic hydrocarbons such as toluene and xylenes, alcohols such as methanol, ethanol, propanol and butanol. The reaction temperature is in the range of ambient temperature (25° C.) to 140° C.

In the second step of preparation of the phenalkamine curing agents, the Mannich base intermediate of step 1 is reacted with the compound with at least one alkylene or aralkylene group and at least two amino groups for the amine exchange to take place. In one embodiment, the process is carried out at temperatures ranging from 80° C. to 150° C. In another embodiment, the process is carried out at 120° C-150° C. In a further embodiment, the process is carried out at 120° C-140° C. During this step the secondary amine used in step 1 is liberated and recovered by condensing it into a vessel at sub-ambient temperature (5° C.).

Preferred examples of compounds with at least one alkylene or aralkylene group and at least two amino groups which are used in step 2 are ethylenediamine (EDA), diethylenetriamine (DETA), triethylenetetramine (TETA), tetraethylenepentamine (TEPA), pentaethylenehexamine (PEHA), hexaethyleneheptamine (HEHA), propylenediamine, dipropylenetriamine, tripropylenetetramine, tetrapropylenepentamine, pentapropylenehexamine, triaminononane, m-xylylenediamine (mXDA), N-(2-aminoethyl)-1, 3-propanediamine (N₃-amine), N,N′-1, 2-ethanediylbis-1, 3-propanediamine (N₄-amine), and N1-{2-[2-(3-Amino-propylamino)-ethylamino]-ethyl}-propane-1,3-diamine (N₅-amine). Other examples include, N-hydroxyethyl ethylenediamine, N-hydroxyethyl diethylenetriamine, N-hydroxyethyl triethylenetetramine, N-hydroxyethyl tetraethylenepentamine, N-hydroxypropyl ethylenediamine, N-hydroxypropyl ethylenediamine, N-hydroxypropyl diethylenetriamine, N-hydroxypropyl triethylenetetramine, and N-hydroxypropyl tetraethylenepentamine. The structures of the hydroxyalkyl amines are shown below.

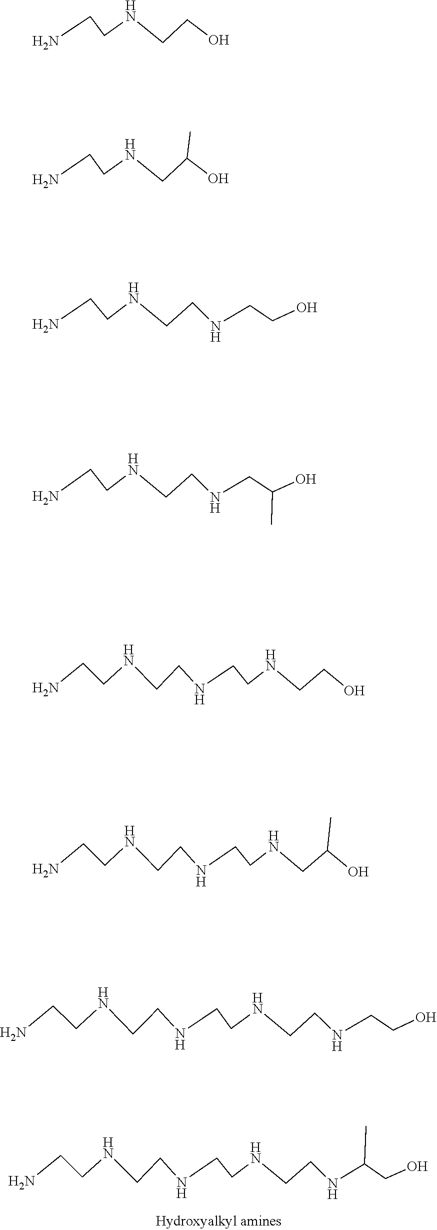

In one embodiment the product viscosity is in the range from 300 mPa·s to 3,000 mPa·s at 25° C. In another embodiment, the product viscosity is in the range from 300 mPa·s to 1,500 mPa·s. In a further embodiment, the product viscosity is in the range from 300 mPa·s to 1,000 mPa·s. This low viscosity is advantageous for using this curing agent in the preparation of epoxy coatings since it requires none or a minimal amount of volatile organic solvent, which may be beneficial for the environment and for the health and safety of workers using these materials.

The present disclosure also provides for novel phenalkamines represented by structures (III) and (VI) below.

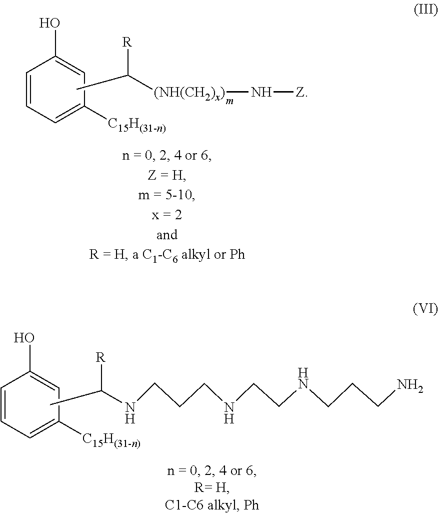

The present disclosure further provides for a curing agent composition comprising a phenalkamine of formula (III) or (VI).

The present disclosure also provides for products produced by the method of making phenalkamines represented by the structures in formulas (III), (IV), (V) and (VI). The present disclosure also provides for the use of these products for the preparation of hardened articles and for the hardening of epoxy resins.

The present disclosure also includes articles of manufacture produced from products as described above. Preferable examples of articles of manufacture are an adhesive, a coating, a primer, a sealant, a curing compound, a construction product, a flooring product, a composite product, laminate, potting compounds, grouts, fillers, cementitious grouts, or self-leveling flooring. Additional components or additives may be used together with the compositions of the present disclosure to produce articles of manufacture. Further, such coatings, primers, sealants, curing compounds or grouts may be applied to metal or cementitious substrates.

The relative amount chosen for the epoxy composition versus that of the curing agent composition, may vary depending upon, for example, the end-use article, its desired properties, and the fabrication method and conditions used to produce the end-use article. For instance, in coating applications using certain amine-epoxy compositions, incorporating more epoxy resin relative to the amount of the curing agent composition may result in coatings which have increased drying time, but with increased hardness and improved appearance as measured by gloss. Amine-epoxy compositions of the present disclosure preferably have stoichiometric ratios of epoxy groups in the epoxy composition to amine hydrogens in the curing agent composition ranging from 1.5:1 to 0.7:1. For example, such amine-epoxy compositions may preferably have stoichiometric ratios of 1.5:1, 1.4:1, 1.3:1, 1.2:1, 1.1:1, 1:1, 0.9:1, 0.8:1, or 0.7:1. In another aspect, the stoichiometric ratio preferably ranges from 1.3:1 to 0.7:1, or from 1.2:1 to 0.8:1, or from 1.1:1 to 0.9:1.

Amine-epoxy compositions of the present disclosure comprise a curing agent composition and an epoxy composition comprising at least one multifunctional epoxy resin. Multifunctional epoxy resin, as used herein, describes compounds containing 2 or more 1,2-epoxy groups per molecule. The epoxy resin is preferably selected from the group consisting of aromatic epoxy resin, alicyclic epoxy resin, aliphatic epoxy resin, glycidyl ester resin, thioglycidyl ether resin, N-glycidyl ether resin, and combinations thereof.

Preferable aromatic epoxy resins suitable for use in the present disclosure preferably comprise the glycidyl ethers of polyhydric phenols, including the glycidyl ethers of dihydric phenols. Further preferred are the glycidyl ethers of resorcinol, hydroquinone, bis-(4-hydroxy-3,5-difluorophenyl)-methane, 1,1-bis-(4-hydroxyphenyl)-ethane, 2,2-bis-(4-hydroxy-3-methylphenyl)-propane, 2,2-bis-(4-hydroxy-3,5-dichlorophenyl) propane, 2,2-bis-(4-hydroxyphenyl)-propane (commercially known as bisphenol A), bis-(4-hydroxyphenyl)-methane (commercially known as bisphenol F, and which may contain varying amounts of 2-hydroxyphenyl isomers), and the like, or any combination thereof. Additionally, advanced dihydric phenols of the following structure also are useful in the present disclosure:

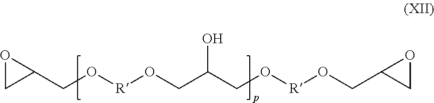

wherein R′ is a divalent hydrocarbon radical of a dihydric phenol, such as those dihydric phenols listed above, and p is an average value between 0 and 7. Materials according to this formula may be prepared by polymerizing mixtures of a dihydric phenol and epichlorohydrin, or by advancing a mixture of a diglycidyl ether of the dihydric phenol and the dihydric phenol. While in any given molecule the value of p is an integer, the materials are invariably mixtures which may be characterized by an average value of p which is not necessarily a whole number. Polymeric materials with an average value of p between 0 and 7 may be used in one aspect of the present disclosure.

In one aspect of the present disclosure at least one multifunctional epoxy resin is preferably a diglycidyl ether of bisphenol-A (DGEBA), an advanced or higher molecular weight version of DGEBA, a diglycidyl ether of bisphenol-F, a diglycidyl ether of novolac resin, or any combination thereof. Higher molecular weight versions or derivatives of DGEBA are prepared by the advancement process, where excess DGEBA is reacted with bisphenol-A to yield epoxy terminated products. The epoxy equivalent weights (EEW) for such products range from 450 to 3000 or more. Because these products are solid at room temperature, they are often referred to as solid epoxy resins.

In preferred embodiments, the at least one multifunctional epoxy resin is the diglycidyl ether of bisphenol-F or bisphenol-A represented by the following structure:

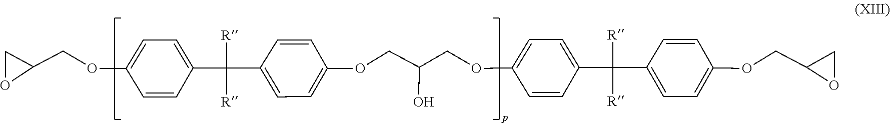

wherein R″═H or CH₃, and p is an average value between 0 and about 7. DGEBA is represented by the above structure when R″═CH₃ and p=0. DGEBA or advanced DGEBA resins are often used in coating formulations due to a combination of their low cost and high performance properties. Commercial grades of DGEBA having an EEW ranging from about 174 to about 250, and more commonly from about 185 to about 195, are readily available. At these low molecular weights, the epoxy resins are liquids and are often referred to as liquid epoxy resins. It is understood by those skilled in the art that most grades of liquid epoxy resin are slightly polymeric, since pure DGEBA has an EEW of about 174. Resins with EEWs between about 250 and about 450, also generally prepared by the advancement process, are referred to as semi-solid epoxy resins because they are a mixture of solid and liquid at room temperature. Preferably, multifunctional resins with EEWs based on solids of about 160 to about 750 are useful in the present disclosure. In another aspect the multifunctional epoxy resin has an EEW in a range from about 170 to about 250.

Preferred examples of alicyclic epoxy compounds are polyglycidyl ethers of polyols having at least one alicyclic ring, or compounds including cyclohexene oxide or cyclopentene oxide obtained by epoxidizing compounds including a cyclohexene ring or cyclopentene ring with an oxidizer. Further preferred are hydrogenated bisphenol A diglycidyl ether; 3,4-epoxycyclohexyl methyl-3,4-epoxycyclohexyl carboxylate; 3,4-epoxy-1-methylcyclohexyl-3,4-epoxy-1-methylhexane carboxylate; 6-methyl-3,4-epoxycyclohexylmethyl-6-methyl-3,4-epoxycyclohexane carboxylate; 3,4-epoxy-3-methylcyclohexylmethyl-3,4-epoxy-3-methylcyclohexane carboxylate; 3,4-epoxy-5-methylcyclohexylmethyl-3,4-epoxy-5-methylcyclohexane carboxylate; bis(3,4-epoxycyclohexylmethyl)adipate; methylene-bis(3,4-epoxycyclohexane); 2,2-bis(3,4-epoxycyclohexyl)propane; dicyclopentadiene diepoxide; ethylene-bis(3,4-epoxycyclohexane carboxylate); dioctyl epoxyhexahydrophthalate; and di-2-ethylhexyl epoxyhexahydrophthalate.

Preferred examples of aliphatic epoxy compounds are polyglycidyl ethers of aliphatic polyols or alkylene-oxide adducts thereof, polyglycidyl esters of aliphatic long-chain polybasic acids, homopolymers synthesized by vinyl-polymerizing glycidyl acrylate or glycidyl methacrylate, and copolymers synthesized by vinyl-polymerizing glycidyl acrylate or glycidyl methacrylate and other vinyl monomers. Further preferred are glycidyl ethers of polyols, such as 1,4-butanediol diglycidyl ether; 1,6-hexanediol diglycidyl ether; a triglycidyl ether of glycerin; a triglycidyl ether of trimethylol propane; a tetraglycidyl ether of sorbitol; a hexaglycidyl ether of dipentaerythritol; a diglycidyl ether of polyethylene glycol; and a diglycidyl ether of polypropylene glycol; polyglycidyl ethers of polyether polyols obtained by adding one type, or two or more types, of alkylene oxide to aliphatic polyols, such as ethylene glycol, propylene glycol, trimethylol propane, and glycerin.

Glycidyl ester resins are obtained by reacting a carboxylic acid compound having at least two carboxyl acid groups in the molecule and epichlorohydrin. Preferred examples of such carboxylic acids include aliphatic, cycloaliphatic, and aromatic carboxylic acids. Further preferred examples of aliphatic carboxylic acids include oxalic acid, succinic acid, glutaric acid, adipic acid, pimelic acid, sebacic acid, suberic acid, azelaic acid, or dimerised or trimerised linoleic acid. Further preferred cycloaliphatic carboxylic acids include tetrahydrophthalic acid, 4-methyltetrahydrophthalic acid, hexahydrophthalic acid or 4-methylhexahydrophthalic acid. Further preferred aromatic carboxylic acids include phthalic acid, isophthalic acid or terephthalic acid.

Thioglycidyl ether resins are derived from dithiols, for example, ethane-1,2-dithiol or bis(4-mercaptomethylphenyl) ether.

N-glycidyl resins are obtained by dehydrochlorination of the reaction products of epichlorohydrin with amines containing at least two amine hydrogen atoms. Such amines are, for example, aniline, n-butylamine, bis(4-aminophenyl)methane, m-xylylenediamine or bis(4-methylaminophenyl)methane. Preferably, the N-glycidyl resins include triglycidyl isocyanurate, N,N′-diglycidyl derivatives of cycloalkylene ureas, e.g., ethylene urea or 1,3-propylene urea, and diglycidyl derivatives of hydantoins, e.g., 5,5-dimethylhydantoin.

For one or more of the embodiments, the resin component further includes a reactive diluent. Reactive diluents are compounds that participate in a chemical reaction with the hardener component during the curing process and become incorporated into the cured composition, and are preferably monofunctional epoxides. Reactive diluents may also be used to vary the viscosity and/or cure properties of the curable compositions for various applications. For some applications, reactive diluents may impart a lower viscosity to influence flow properties, extend pot life and/or improve adhesion properties of the curable compositions. For example, the viscosity may be reduced to allow an increase in the level of pigment in a formulation or composition while still permitting easy application, or to allow the use of a higher molecular weight epoxy resin. Thus, it is within the scope of the present disclosure for the epoxy component, which comprises at least one multifunctional epoxy resin, to preferably further comprise a monofunctional epoxide. Preferred examples of monoepoxides are styrene oxide, cyclohexene oxide and the glycidyl ethers of phenol, cresols, tert-butylphenol, other alkyl phenols, butanol, 2-ethylhexanol, C4 to C14 alcohols, and the like, or combinations thereof. The multifunctional epoxy resin may also be present in a solution or emulsion, with the diluent being water, an organic solvent, or a mixture thereof. The amount of multifunctional epoxy resin may range from 50% to 100%, 50% to 90%, 60% to 90%, 70% to 90%, and in some cases 80% to 90%, by weight, of the epoxy component. For one or more of the embodiments, the reactive diluent is less than 60 weight percent of a total weight of the resin component.

Preferably suitable multifunctional epoxy compounds are the diglycidyl ethers of bisphenol-A and bisphenol-F, the advanced diglycidyl ethers of bisphenol-A and bisphenol-F, and the epoxy novolac resins. The epoxy resin may be a single resin, or it may be a mixture of mutually compatible epoxy resins.

Compositions of the present disclosure may be used to produce various articles of manufacture. Depending on the requirements during the manufacturing of or for the end-use application of the article, various additives may be employed in the formulations and compositions to tailor specific properties. Preferred examples of additives are solvents (including water), accelerators, plasticizers, fillers, fibers, such as glass or carbon fibers, pigments, pigment dispersing agents, rheology modifiers, thixotropes, flow or leveling aids, surfactants, defoamers, biocides, or any combination thereof. It is understood that other mixtures or materials that are known in the art may be included in the compositions or formulations and are within the scope of the present disclosure.

Preferred examples of articles in accordance with the present disclosure are a coating, an adhesive, a construction product, a flooring product, or a composite product. Coatings based on these amine-epoxy compositions may be solvent-free or may contain diluents, such as water or organic solvents, as needed for the particular application. Coatings may contain various types and levels of pigments for use in paint and primer applications. In one embodiment, amine-epoxy coating compositions comprise a layer having a thickness ranging from 25 to 500 μm (micrometer) for use in a protective coating applied onto metal substrates. In another embodiment, the amine-epoxy coating compositions comprise a layer having a thickness ranging from 80 to 300 μm for use in a protective coating applied onto metal substrates. In a further embodiment, the amine-epoxy coating compositions comprise a layer having a thickness ranging from 100 to 250 μm for use in a protective coating applied onto metal substrates. In addition, for use in a flooring product or a construction product, coating compositions preferably comprise a layer having a thickness ranging from 50 to 10,000 μm, depending on the type of product and the required end-properties. A coating product that delivers limited mechanical and chemical resistances comprises a layer having a thickness ranging from 50 to 500 μm, preferably 100 to 300 μm; whereas a coating product, such as, for example, a self-leveling floor that delivers high mechanical and chemical resistances comprises a layer having a thickness ranging from 1,000 to 10,000 μm, preferably 1,500 to 5,000 μm.

Various substrates are suitable for the application of coatings of this invention with proper surface preparation, as is well known to one of ordinary skill in the art. Preferable substrates are concrete and various types of metals and alloys, such as steel and aluminum. Coatings of the present disclosure are suitable for the painting or coating of large metal objects including ships, bridges, industrial plants and equipment, or cementitious substrates such as industrial floors.

Coatings of this invention may be applied by any number of techniques including spray, brush, roller, paint mitt, and the like. In order to apply very high solids content or 100% solids coatings of this invention, plural component spray application equipment may be used, in which the amine and epoxy components are mixed in the lines leading to the spray gun, in the spray gun itself, or by mixing the two components together as they leave the spray gun. Using this technique may alleviate limitations with regard to the pot life of the formulation, which typically decreases as both the amine reactivity and the solids content increases. Heated plural component equipment may be employed to reduce the viscosity of the components, thereby improving ease of application.

Construction and flooring applications include compositions comprising the amine-epoxy compositions of the present disclosure in combination with concrete or other materials commonly used in the construction industry. Preferable applications of compositions of the present disclosure are its use as a primer, a deep penetrating primer, a coating, a curing compound, and/or a sealant for new or old concrete, such as referenced in ASTM C309-97, which is incorporated herein by reference. As a primer or a sealant, the amine-epoxy compositions of the present disclosure may be applied to surfaces to improve adhesive bonding prior to the application of a coating. As it pertains to concrete and cementitious application, a coating is an agent used for application on a surface to create a protective or decorative layer or a coat. Crack injection and crack filling products also may be prepared from the compositions disclosed herein. Amine-epoxy compositions of the present disclosure may be mixed with cementitious materials, such as concrete mix, to form polymer or modified cements, tile grouts, and the like. Non-limiting examples of composite products or articles comprising amine-epoxy compositions disclosed herein include tennis rackets, skis, bike frames, airplane wings, glass fiber reinforced composites, and other molded products.

In a particular use of the curing agent compositions of the present disclosure, coatings may be applied to various substrates, such as concrete and metal surfaces at low temperature, with fast cure speed and good coating appearance. This is especially important for top-coat application where good aesthetics is desired, and provides a solution to a long-standing challenge in the industry where fast low-temperature cure with good coating appearance remains to be overcome. With fast low-temperature cure speed, the time of service or where equipment is down may be shortened, or for outdoor applications, the work season may be extended in cold climates.

Fast epoxy curing agents enable amine-cured epoxy coatings to cure in a short period of time with a high degree of cure. The cure speed of a coating is monitored by thin film set time (TFST) which measures the time period a coating dries. The thin film set time is categorized in 4 stages: phase 1, set to touch; phase 2, tack free: phase 3, dry hard; and phase 4, dry through. The phase 3 dry time is indicative of how fast a coating cures and dries. For a fast ambient cure coating, phase 3 dry time is less than 6 hours, or less than 4 hours, or preferred to be less than 4 hours. Low temperature cure typically refers to cure temperature below ambient temperature, 10° C. or 5° C., or 0° C. in some cases. For a fast low temperature cure, phase 3 dry time at 5° C. is less than 15 hours, or less than 12 hours, or less than 10 hours.

How well a coating cures is measured by the degree of cure. Degree of cure is often determined by using DSC (differential scanning calorimetry) technique which is well-known to those skilled in the art. A coating which cures thoroughly will have a degree of cure at ambient temperature (25° C.) of at least 85%, or at least 90%, or at least 95% after 7 days, and at least 80%, or at least 85%, or at least 90% at 5° C. after 7 days.

Many of the fast low temperature epoxy curing agents may cure an epoxy resin fast. However due to poor compatibility of the epoxy resin and curing agents especially at low temperature of 10° C. or 5° C., there is phase separation between resin and curing agent and curing agent migrating to coating surface, resulting in poor coating appearance manifested as sticky and cloudy coatings. Good compatibility between epoxy resin and curing agent leads to clear glossy coating with good carbamation resistance and good coating appearance. The curing agent compositions of the present disclosure offers the combination of fast cure speed, good compatibility and high degree of cure.

In another aspect of this invention the phenalkamine curing agent of this invention may be used in combination with another amine curing agent (as a co-curing agent) for curing epoxy resins. Hence, the amine-epoxy composition, in accordance with the present disclosure, comprises:
(a) a curing agent composition comprising at least one of the phenalkamine compositions of this invention shown below:

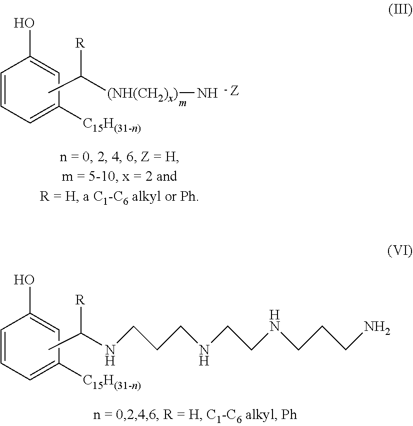

(b) an epoxy composition comprising at least one multifunctional epoxy resin as described above; and
(c) an amine co-curing agent having at least two amine functionalities.

Preferable examples of amine co-curing agents include diethylenetriamine (DETA), triethylenetetramine (TETA), teraethylenepentamine (TEPA), pentaethylenehexamine (PEHA), hexamethylenediamine (HMDA), 1,3-pentanediamine (DYTEK™EP), 2-methyl-1,5-pentanediamine (DYTEK™A) N-(2-aminoethyl)-1, 3-propanediamine (N-3-Amine), N,N′-1, 2-ethanediylbis-1, 3-propanediamine (N4-amine), or dipropylenetriamine; an arylaliphatic amine such as m-xylylenediamine (mXDA), or p-xylylenediamine; a cycloaliphatic amine such as 1,3-bisaminocyclohexylamine (1,3-BAC), isophorone diamine (IPDA), or 4,4′-methylenebiscyclohexanamine; an aromatic amine such as m-phenylenediamine, diaminodiphenylmethane (DDM), or diaminodiphenylsulfone (DDS); a heterocyclic amine such as N-aminoethylpiperazine (NAEP), or 3,9-bis(3-aminopropyl)2, 4,8, 10-tetraoxaspiro (5,5)undecane; an alkoxyamine where the alkoxy group can be an oxyethylene, oxypropylene, oxy-1, 2- butylene, oxy-1, 4-butylene or co-polymers thereof such as 4,7-dioxadecane-1,10-diamine, I-propanamine, 3,3′-(oxybis (2,1-ethanediyloxy))bis(diaminopropylated diethylene glycol ANCAMINE1922A),poly(oxy(methyl-1, 2-ethanediyl)), alpha-(2-aminomethylethyl)omega-(2-aminomethylethoxy) (JEFFAMINE D 230, D-400), triethyleneglycoldiamine and oligomers (JEFFAMINEXTJ-504, JEFFAMINE XTJ-512), poly(oxy(methyl-1, 2-ethanediyl)), alpha, alpha′-(oxydi-2, 1-ethanediyl)bis(omega-(aminomethylethoxy)) (JEFFAMINE XTJ-511), bis(3-aminopropyl)polytetrahydrofuran 350, bis(3-aminopropyl)polytetrahydro furan 750, poly(oxy(methyl-1, 2-ethanediyl)), a-hydro-w-(2-aminomethylethoxy)ether with 2-ethyl-2-(hydroxymethyl)-1, 3-propanediol (3:I) (JEFFAMINE T-403),and diaminopropyldiaminopropyl dipropylene glycol.

Other amine co-curing agents include amidoamine and polyamide curing agents. Polyamide curing agents are comprised of the reaction products of dimerized fatty acid (dimer acid) and amine compounds having at least two ethylene groups, and usually a certain amount of monomeric fatty acid which helps to control molecular weight and viscosity. “Dimerized” or “dimer” or “polymerized” fatty acid refers, preferably, to polymerized acids obtained from unsaturated fatty acids. They are described more fully in T. E. Breuer, ‘Dimer Acids’, in J. I. Kroschwitz (ed.), Kirk-Othmer Encyclopedia of Chemical Technology, 4′ Ed., Wley, New York, 1993,Vol. 8, pp. 223-237. Common mono-functional unsaturated C-6 to C-20 fatty acids also employed in making polyamides include tall oil fatty acid (TOFA) or soya fatty acid or the like.

Other amine co-curing agents include phenalkamines and Mannich bases of phenolic compounds with amines and formaldehyde.

In one embodiment, the weight ratio of the phenalkamine curing agent of this composition and the amine co-curing agent is 1:1 to 1:0.05. In another embodiment, the weight ratio of the phenalkamine curing agent of this composition and the amine co-curing agent is 1:0.75 to 1:0.25.

The combined phenalkamine curing agent composition of this invention and the amine co-curing agent and epoxy compositions of the present disclosure preferably have stoichiometric ratios of epoxy groups in the epoxy composition to amine hydrogens in the curing agent composition ranging from 1.5:1 to 0.7:1. For example, such amine-epoxy compositions may preferably have stoichiometric ratios of 1.5:1, 1.4:1, 1.3:1, 1.2:1, 1.1:1, 1:1, 0.9:1, 0.8:1, or 0.7:1. In another aspect, the stoichiometric ratio ranges from 1.3:1 to 0.7:1, or from 1.2:1 to 0.8:1, or from 1.1:1 to 0.9:1.

The following invention is directed to the following aspects:
<1> A method for producing a phenalkamine comprising the steps of a. preparing a Mannich base by reacting cardanol with a secondary amine of formula

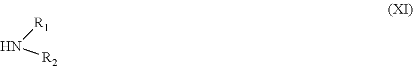

with R₁ and R₂ being, independently of each other, a C₁-C₆ alkyl or aryl group, and an aldehyde, and
b. reacting the Mannich base with a compound with at least one alkylene or aralkylene group and at least two amino groups.
   This method for producing a phenalkamine has the advantage that the molecular weight distribution of the products can be controlled better, so that it is easier to achieve products with lower viscosity. Another advantage is that phenalkamines can be produced that cannot be produced by a direct Mannich reaction, e.g. when cyclization reactions occur.
<2.> A preferred method according to aspect <1>, wherein the secondary amine is dimethylamine.
<3.> A preferred method according to aspect <1> or aspect <2>, wherein the compound with at least one alkylene or aralkylene group is represented by the following formula (VIII)

NH₂((CH₂),NH)−Z x=2-10, m=1-10, Z═H, (CH₂)ₚOH p=2,3 (VIII)
<4.> A method according to aspect <1> or aspect <2>,
   wherein the compound has the following formula (IX)

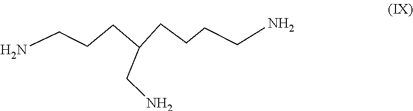 [SMILES: NCCCCC(CN)CCCN]

<5.> A method according to aspect <1> or aspect <2>,
   wherein the compound has the following formula (X)

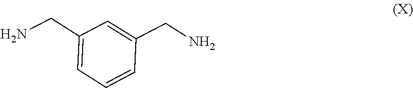

<6.> A method according to aspect <3>, wherein the compound of formula (VIII) is a mixture of triethylenetetramine, tetraethylenepentamine, hydroxyethyl diethylenetriamine and hydroxyethyl triethylenetetramine.
<7.> A method according to aspect <3>, wherein in formula (VIII) Z═H, m=5-10 and x=2.
<8.> A method according to aspect <3>, wherein the compound of formula (VIII) is N,N′-1,2-ethanediyl-bis-1,3-propanediamine.
<9.> A product produced by a process according to one of aspects <1> to <8>. As can be seen from the experiments, e.g. from example 12 in comparison with example 15, the MXDA phenalkamine produced according to the invention has a much lower viscosity.
<10.> Use of the products according to aspect <9> for the preparation of hardened articles. Preferable articles are selected from coatings, adhesives, primers, sealants, curing compounds, construction products, flooring products, or composite products.
<11.> Use of the products according to aspect <5> for the hardening of epoxy resins.
<12.> A phenalkamine of formula (III)

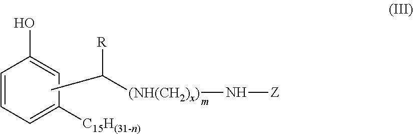

with n=0, 2, 4 or 6, Z═H, m=5-10, x=2 and R═H, a C₁-C₆ alkyl or Ph.
<13.> A phenalkamine of formula (VI)

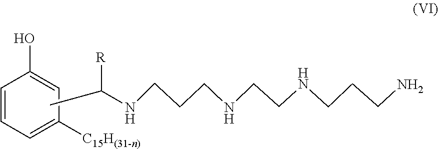

with n=0, 2, 4 or 6, and R═H, a C₁-C₆ alkyl or Ph.
According to the present invention, this compound could previously not be synthesized via the regular Mannich reaction route.

The invention is further illustrated by the following examples, which are not to be construed in any way as imposing limitations to the scope of this invention. Various other aspects, embodiments, modifications, and equivalents thereof which, after reading the description herein, may suggest themselves to one of ordinary skill in the art without departing from the spirit of the present invention or the scope of the appended claims.

### EXAMPLES

These Examples are provided to demonstrate certain aspects of the invention and shall not limit the scope of the claims appended hereto.

### Example 1

### Preparation of Cardanol/Dimethylamine Mannich Base Intermediate in Parr Pressure Reactor

Cardanol (298.46 g, 1 mole) and 40% aqueous dimethylamine (112.7 g, 2.5 moles, 281.75 g of 40% aqueous solution) were charged to a 2-L Parr pressure reactor. The reactor contents were purged 3× with N₂, venting down to ambient pressure afterwards. The mixture was stirred to 300 rpm while a 37% aqueous formaldehyde solution (75.07 g, 2.5 moles, 202.7g of 37% aqueous solution) was added via a pump over a half hour while maintaining the temperature at 25° C. After formaldehyde addition the temperature was increased to 140° C., while monitoring the pressure rise. The temperature was maintained at 140° C. for 1 h and pressure of ˜100 psi. The reactor was cooled to room temperature and the contents poured into a 2L flask. The water was removed by distillation to recover the product as a reddish brown liquid.

### Example 2

### Preparation of Cardanol/Dimethylamine Mannich Base Intermediate in Glass Reactor

Cardanol (298.46 g, 1 mole) and 40% aqueous dimethylamine (45 g, 1.0 mole, 112.5 g of 40% aqueous solution) were charged to a 3-neck glass reactor equipped with a N₂ inlet tube, a thermocouple, condenser and addition funnel. The reactor contents were purged with N₂. The mixture was stirred with an over-head mechanical stirrer and heated to 50° C. A 37% aqueous formaldehyde solution (30 g, 1.0 mole, 81 g of 37% aqueous solution) was added over a half hour while maintaining the temperature at 50-70° C. After formaldehyde addition, the temperature was kept at 80-90° C. The mixture was maintained at this temperature for 1 h. The reactor was cooled to room temperature and the contents poured into a 2L flask. The water was removed by distillation to recover the product as a reddish brown liquid.

### Example 3

### Preparation of Ethylenediamine Derived Phenalkamine via the Amine-Exchange Process from the Cardanol/Dimethylamine Mannich Base Intermediate

The cardanol/dimethylamine Mannich base intermediate (461.5 g,1.3 mole) from example 1 and ethylenediamine (78 g, 1.3 mole) were charged to a 2-liter glass reactor equipped with a thermocouple, nitrogen inlet tube, an overhead stirrer, and an adapter with a gas outlet tube. The top of the gas outlet tube was attached to a dry-ice cold trap, and the bottom of the adapter was connected to a round-bottom flask containing a 50% aqueous acetic acid solution cooled by an ice-bath. The reaction was heated up to 140° C. and kept at this temperature for 3 h. The dimethylamine (DMA) which evolved was condensed by the dry-ice trap and collected in the cold acetic acid solution. Approximately 1.3 mole of DMA was collected. The product obtained was a light brown liquid.

### Example 4

### Preparation of Ethylenediamine Derived Phenalkamine via the Amine-Exchange Process from the Cardanol/Dimethylamine Mannich Base Intermediate of Example 2

The cardanol/dimethylamine Mannich base intermediate (355 g,1.0 mole) from example 2 and ethylenediamine (60 g, 1.0 mole) were charged to a 2-liter glass reactor equipped with a thermocouple, nitrogen inlet tube, an overhead stirrer, and an adapter with a gas outlet tube. The top of the gas outlet tube was attached to a dry-ice cold trap, and the bottom of the adapter was connected to a round-bottom flask containing a 50% aqueous acetic acid solution cooled by an ice-bath. The reaction was heated up to 140° C. and kept at this temperature for 3 h. The dimethylamine (DMA) which evolved was condensed by the dry-ice trap and collected in the cold acetic acid solution. Approximately 1.0 mole of DMA was collected. The product obtained was a light brown liquid.

### Example 5

### Preparation of Diethylenetriamine Derived Phenalkamine Via the Amine-Exchange Process from the Cardanol/Dimethylamine Mannich Base Intermediate.

The cardanol/dimethylamine Mannich base intermediate (461.5 g,1.3 mole) from example 1 and diethylenetriamine (134,12 g, 1.3 mole) were charged to a 2-liter glass reactor equipped with a thermocouple, nitrogen inlet tube, an overhead stirrer, and an adapter with a gas outlet tube. The top of the gas outlet tube was attached to a dry-ice cold trap, and the bottom of the adapter was connected to a round-bottom flask containing a 50% aqueous acetic acid solution cooled by an ice-bath. The reaction was heated up to 140° C. and kept at this temperature for 3 h. The dimethylamine (DMA) which evolved was condensed by the dry-ice trap and collected in the cold acetic acid solution. Approximately 1.3 mole of DMA was collected. The product obtained was a light brown liquid.

### Example 6

### Preparation of XA-70* derived Phenalkamine Via the Amine-Exchange Process from the Cardanol/Dimethylamine Mannich Base Intermediate

The cardanol/dimethylamine Mannich base intermediate (461.5 g,1.3 mole) from example 1 and XA-70 (200.2 g, 1.3 mole) were charged to a 2-liter glass reactor equipped with a thermocouple, nitrogen inlet tube, an overhead stirrer, and an adapter with a gas outlet tube. The top of the gas outlet tube was attached to a dry-ice cold trap, and the bottom of the adapter was connected to a round-bottom flask containing a 50% aqueous acetic acid solution cooled by an ice-bath. The reaction was heated up to 140° C. and kept at this temperature for 3 h. The dimethylamine (DMA) which evolved was condensed by the dry-ice trap and collected in cold the acetic acid solution. Approximately 1.3 mole of DMA was collected. The product obtained was a light brown liquid.

*XA-70 is a mixture of triethylene tetramine and tetraethylenepentamine (-55 wt. %) and hydroxyethylamines composed of hydroxyethyl diethylenetriamine, hydroxyethyl triethylenetetramine and lower alkanolamines (total hydroxyethylamines, ˜45 wt. %) with avg. molecular weight of 154, available from Akzo corp.

### Example 7

### Preparation of *ECA-29 Derived Phenalkamine Via the Amine-Exchange Process from the Cardanol/Dimethylamine Mannich Base Intermediate

The cardanol/dimethylamine Mannich base intermediate (461.5 g,1.3 mole) from example 1 and ECA-29 (325 g, 1.3 mole) were charged to a 2-liter glass reactor equipped with a thermocouple, nitrogen inlet tube, an overhead stirrer, and an adapter with a gas outlet tube. The top of the gas outlet tube was attached to a dry-ice cold trap, and the bottom of the adapter was connected to a round-bottom flask containing a 50% aqueous acetic acid solution cooled by an ice-bath. The reaction was heated up to 140° C. and kept at this temperature for 3 h. The dimethylamine (DMA) which evolved was condensed by the dry-ice trap and collected in the cold acetic acid solution. Approximately 1.3 mole of DMA was collected. The product obtained was a light brown liquid.

*ECA-29 is a mixture of oligomeric polyethylene amines with Avg. M.Wt of 250 available from Huntsmann Corp.

### Example 8

### Preparation of N,N′-1,2-Ethanediylbis-1,3-Propanediamine (N₄-Amine) Derived Phenalkamine Via the Amine-Exchange Process from the Cardanol/Dimethylamine Mannich Base Intermediate

The cardanol/dimethylamine Mannich base intermediate (461.5 g,1.3 mole) from example 1 and N₄-amine (226.2 g, 1.3 mole) were charged to a 2-liter glass reactor equipped with a thermocouple, nitrogen inlet tube, an overhead stirrer, and an adapter with a gas outlet tube. The top of the gas outlet tube was attached to a dry-ice cold trap, and the bottom of the adapter was connected to a round-bottom flask containing a 50% aqueous of acetic acid solution cooled by an ice-bath. The reaction was heated up to 140° C. and kept at this temperature for 3 h. The dimethylamine (DMA) which evolved was condensed by the dry-ice trap and collected in the cold acetic acid solution. Approximately 1.3 mole of DMA was collected. The product obtained was a light brown liquid.

### Example 9

### Preparation of N,N′-1,2-Ethanediylbis-1,3-Propanediamine (N₄-Amine) Derived Phenalkamine Via the Amine-Exchange Process from the Cardanol/Dimethylamine Mannich Base Intermediate of Example 2

The cardanol/dimethylamine Mannich base intermediate (355 g,1.0 mole) from example 2 and N₄-amine (174 g, 1.0 mole) were charged to a 2-liter glass reactor equipped with a thermocouple, nitrogen inlet tube, an overhead stirrer, and an adapter with a gas outlet tube. The top of the gas outlet tube was attached to a dry-ice cold trap, and the bottom of the adapter was connected to a round-bottom flask containing a 50% aqueous acetic acid solution cooled by an ice-bath. The reaction was heated up to 140° C. and kept at this temperature for 3 h. The dimethylamine (DMA) which evolved was condensed by the dry-ice trap and collected in the cold acetic acid solution. Approximately 1.0 mole of DMA was collected. The product obtained was a light brown liquid.

### Example 10

### Preparation of Triaminononane Derived Phenalkamine Via the Amine-Exchange Process from the Cardanol/Dimethylamine Mannich Base Intermediate of Example 1

The cardanol/dimethylamine Mannich base intermediate (461.5 g,1.0 mole) from example 1 and triaminononane (225.29 g, 1.3 mole) were charged to a 2-liter glass reactor equipped with a thermocouple, nitrogen inlet tube, an overhead stirrer, and an adapter with a gas outlet tube. The top of the gas outlet tube was attached to a dry-ice cold trap, and the bottom of the adapter was connected to a round-bottom flask containing a 50% aqueous acetic acid solution cooled by an ice-bath. The reaction was heated up to 140° C. and kept at this temperature for 3 h. The dimethylamine (DMA) which evolved was condensed by the dry-ice trap and collected in the cold acetic acid solution. Approximately 1.3 mole of DMA was collected. The product obtained was a light brown liquid.

### Example 11

### Preparation of Triaminononane Derived Phenalkamine Via the Amine-Exchange Process from the Cardanol/Dimethylamine Mannich Base Intermediate of Example 2

The cardanol/dimethylamine Mannich base intermediate (355 g,1.0 mole) from example 2 and triaminononane (173.3 g, 1.0 mole) were charged to a 2-liter glass reactor equipped with a thermocouple, nitrogen inlet tube, an overhead stirrer, and an adapter with a gas outlet tube. The top of the gas outlet tube was attached to a dry-ice cold trap, and the bottom of the adapter was connected to a round-bottom flask containing a 50% aqueous acetic acid solution cooled by an ice-bath. The reaction was heated up to 140° C. and kept at this temperature for 3 h. The dimethylamine (DMA) which evolved was condensed by the dry-ice trap and collected in the cold acetic acid solution. Approximately 1.0 mole of DMA was collected. The product obtained was a light brown liquid.

### Example 12

### Preparation of m-Xylenediamine Derived Phenalkamine Via the Amine-Exchange Process from the Cardanol/Dimethylamine Mannich Base Intermediate of Example 1

The cardanol/dimethylamine Mannich base intermediate (461.5 g,1.0 mole) from example 1 and m-xylenediamine (177.06 g, 1.3 mole) were charged to a 2-liter glass reactor equipped with a thermocouple, nitrogen inlet tube, an overhead stirrer, and an adapter with a gas outlet tube. The top of the gas outlet tube was attached to a dry-ice cold trap, and the bottom of the adapter was connected to a round-bottom flask containing a 50% aqueous acetic acid solution cooled by an ice-bath. The reaction was heated up to 140° C. and kept at this temperature for 3 h. The dimethylamine (DMA) which evolved was condensed by the dry-ice trap and collected in the cold acetic acid solution. Approximately 1.3 mole of DMA was collected. The product obtained was a light brown liquid.

### Example 13

### Preparation of m-Xylenediamine Derived Phenalkamine Via the Amine-Exchange Process from the Cardanol/Dimethylamine Mannich Base Intermediate of Example 2

The cardanol/dimethylamine Mannich base intermediate (355 g,1.0 mole) from example 2 and m-xylenediamine (136.2 g, 1.0 mole) were charged to a 2-liter glass reactor equipped with a thermocouple, nitrogen inlet tube, an overhead stirrer, and an adapter with a gas outlet tube. The top of the gas outlet tube was attached to a dry-ice cold trap, and the bottom of the adapter was connected to a round-bottom flask containing a 50% aqueous acetic acid solution cooled by an ice-bath. The reaction was heated up to 140° C. and kept at this temperature for 3 h. The dimethylamine (DMA) which evolved was condensed by the dry-ice trap and collected in the cold acetic acid solution. Approximately 1.0 mole of DMA was collected. The product obtained was a light brown liquid.

### Example 14

### Attempted Preparation of N,N′-1,2-Ethanediylbis-1,3-Propanediamine (N₄-Amine) Derived Phenalkamine Via the Direct Mannich Process

N,N′-1,2-ethanediylbis-1,3-propanediamine (N₄ amine) (226.2 g) and cardanol (298 g, 1 mole) were charged into a 3-neck glass reactor equipped with a N₂ inlet tube, a thermocouple, condenser and addition funnel. The reactor contents were purged with N₂. The mixture was stirred with an over-head mechanical stirrer and heated to 85° C. A 37% aqueous formaldehyde solution (39 g, 1.3 mole, 105.4 g of 37% aqueous solution) was added over a half hour while maintaining the temperature at 85-95° C. After formaldehyde addition, the temperature was kept at 85-90° C. for 1 h. The mixture was cooled to 50° C. and water was removed by distillation in-vacuo. The product was a mixture of the monocyclized and di-cyclized amine containing compounds shown below.

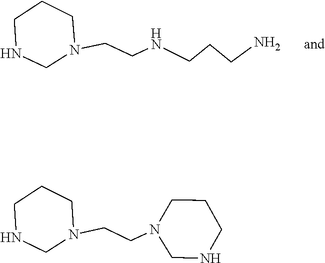 [SMILES: NCCCNCCN1CCCNC1 ; C1CNCN(CCN2CCCNC2)C1]

### Example 15

### Preparation of m-Xylenediamine Derived Phenalkamine Via the Standard Mannich Base Approach

m-xylenediamine (177.06 g, 1.3 mole) and cardanol (298 g, 1 mole) were charged into a 3-neck glass reactor equipped with a N₂ inlet tube, a thermocouple, condenser and addition funnel. The reactor contents were purged with N₂. The mixture was stirred with an over-head mechanical stirrer and heated to 85° C. A 37% aqueous formaldehyde solution (39 g, 1.3 mole, 105.4 g of 37% aqueous solution) was added over a half hour while maintaining the temperature at 85-95° C. After formaldehyde addition, the temperature was kept at 85-90° C. for 1 h. The mixture was cooled to 50° C. and water was removed by distillation in-vacuo.

### Evaluation Of Examples

In order to demonstrate the novelty of this invention, curing agents selected from Examples 3-15, were evaluated for use as two component epoxy coatings. Coatings of amine-epoxy compositions were prepared and tested as follows. Curing agent compositions, including individual amine compositions in accordance with the present invention were prepared by contacting and mixing the components given in the tables that follow. The respective curing agent hardener was then mixed with a multifunctional epoxy resin at the use level indicated in the tables in parts per hundred weight resin (PHR). The epoxy resin used in these examples was either the diglycidyl ether of bisphenol-A (DGEBA), grade D.E.R.™ 331 or Epon™ 828 with an EEW in the range of 182 to 192. These epoxy resins are commercially available from the Dow Chemical Company and Hexion respectively. Two comparative examples C1 & C2 were also screened, they are the commercially available phenalkamines, Sunmide CX-105 and Sunmide CX-101 based on the amines ethylenediame (EDA) and diethylentriame (DETA), available from Evonik Corp. Example 14 demonstrates employing the standard cardanol-formaldehyde-amine synthesis route with a long chain N,N′-1, 2-ethanediylbis-1, 3-propanediamine (N₄-amine) does not result in the formation of a phenalkamine curing agent. Example 15 acts as a comparative example, showing this is a phenalkamine produced via the standard cardanol-formaldehyde-amine route based on m-xylenediamine (MXDA). In Examples 3, 5, 6, 7, 8, 11, 12, 15 and the comparative commercial sample examples (C1, C2), clear coatings were applied to standard glass panels to produce samples for drying time testing using a Beck-Koller drying time recorder and for hardness development by the Persoz pendulum hardness method. Clear coatings for surface appearance assessment, waterspot and resistance to carbamation were applied to uncoated, Lenata charts. Coatings were applied at about 150 μm WFT (wet film thickness) using a Bird bar applicator resulting in dry film thicknesses ranging from approximately 120 μm to 140 μm. Coatings of Examples 3, 5, 6, 7, 8, 11, 12, 15, C1 & C2 were cured either at 5° C. and 80% RH (relative humidity), or 25° C. and 60% RH, using a Weiss climate chamber (type WEKK0057.S). Persoz Hardness was measured at the times indicated in the tables. Clear coatings for impact resistance and mandrel bend testing were applied to smooth finished cold-rolled steel test panels, (approximate size 76 mm×152 mm×0. 5 mm thick), using a nominal 150 μm WFT wire bar. Metal test panels were obtained from Q Panel Lab Products. Coating properties were measured in accordance with the standard test methods listed in Table 1. Waterspot resistance was tested by placing drops of water on the surface of the coating for a specified time and observing the impact on the coating. This test is used in the industry to determine if the surface of the coating is damaged or aesthetically impacted by extended contact with water or moisture. Carbamation resistance was tested on clear coatings following cure at both 23° C. and 50% relative humidity, and 5° C. and 80% relative humidity, for 1 day and 7 days. A lint free cotton patch was placed on the test panel, ensuring that it was at least 12 mm from the edge of the panel. The cotton patch was dampened with 2-3 ml of de-mineralized water and covered with a watch glass. The panel was left undisturbed for the specified time (standard time is 24 h). After which time the patch was removed and the coating was dried with a cloth or tissue. The panel was examined immediately for carbamation and rated according to the ratings listed in Table 1. The gel time characterizes the time a composition transitions from a liquid to a gel and is an indication of the practical working pot life of the coating system. The gel time of the amine-epoxy compositions was measured with a TECHNE gelation timer model GT-5 using ASTM D2471.

TABLE 1
Test Methods

| Property | Response | Test Method |
|---|---|---|
| Gel time | Sample Gelation (150 | D2471 |
| | gram mixture) | |
| Drying time: BK | Thin film set times phases | ASTM D5895 |
| recorder | 2 & 3 (hour) | |
| Specular gloss | Gloss at 20° and 60° | ASTM D523 |
| Persoz pendulum | Persoz hardness (s) | ASTM D4366 |
| hardness | | |
| Mechanical | Conical bending | ASTM D522 |
| property | Impact Resistance, (falling | ASTM G14 |
| | weight test) | |
| | cm · kg | |
| Carbamation | Surface whitening due to | P: Poor: White surface |
| | amine-reaction with water | F: Fair: Slight whitening |
| | & atmospheric CO₂ | G: Good: Slight Haze |
| | | Ex: Very good: Glossy film |

### Assessment of Basic Handling Performance Properties:

The curing agents from this invention were assessed for base handling properties, including the viscosity and appearance. Properties are summarized in Table 2.

TABLE 2
Phenalkamine Curing Agent Handling Properties

| Property | Ex 3 | Ex 5 | Ex 6 | Ex 7 | Ex 8 | Ex11 | Ex 12 | Ex 15 | C1 | C2 |
|---|---|---|---|---|---|---|---|---|---|---|
| Base Amine¹ | EDA | DETA | XA70 | ECA29 | N4 | TAN | MXDA | MXDA | EDA | DETA |
| Viscosity | 780 | 1,850 | 2,930 | 2,900 | 834 | 850 | 716 | 3,200 | 28,000 | 40,000 |
| mPa · s | | | | | | | | | | |
| AHEW | 112 | 114 | 107 | 91 | 90 | 97 | 142 | 142 | 130 | 115 |
| Loading | 60 | 60 | 57 | 49 | 47 | 51 | 75 | 75 | 68 | 60* |
| PHR | | | | | | | | | | |

| | |
|---|---|
| Appearance | All dark amber Colour > Gardner 12 < Gardner 16 |

¹Base amine used in the synthesis of the phenalkamine-mannich base curative
*Actual loading used 81phr, due to xylene (20%) was added to CX-101 to achieve suitable handling viscosity

Products based on the amine exchange reaction of a cardanol derived mannich base with a compound with at least one alkylene or aralkylene group and at least two amino groups have the advantage of providing amine epoxy hardeners with low initial viscosity. Examples 3 & 5 are phenalkamines manufactured via the exchange process using EDA & DETA respectively, which exhibit significantly reduced handling viscosities vs phenalkamines containing these amines produced via the conventional cardanol-formaldehyde-amine condensation process as illustrated by comparative examples C1 & C2. In the case of Example 8, this is a phenalkamine based on the exchange reaction of the cardanol-DMA phenolic with N,N′-1,2-ethanediylbis-1,3-propanediamine (N₄ amine). The resultant reaction product is a low viscosity phenalkamine <1000 mPa·s. Attempts to synthesize a phenalkamine from the N,N′-1,2-ethanediylbis-1,3-propanediamine (N₄ amine) via the direct route (Example 14), proved unsuccessful, due to the competing N4 amine-formaldehyde cyclization reaction. Example 8, therefore represents a novel and practical route to the manufacture of phenalkamines from this class of long chain compound with at least one alkylene or aralkylene group and at least two amino groups. Example 12 and Example 15 are phenalkamines based on the arylaliphatic amines compound, m-xylenediamine (MXDA), synthesized by the cardanol-DMA-amine (exchange) and cardanol-formaldehyde-amine (direct) methods respectively. The data in Table 2 highlights the lower curing agent viscosity obtained utilizing the exchange process.

Coatings Made from the Amine Epoxy Hardeners

Performance properties obtained for clear coatings formulated with several new phenalkamine chosen from Examples 3-15 and the comparative examples C1& C2 are illustrated in Table 3.

TABLE 3
Test summary of gel time, dry time, Persoz hardness
and surface appearance properties of clear coatings

| Property | Ex 3 | Ex 5 | Ex 6 | Ex 7 | Ex 8 | Ex11 | Ex 12 | Ex 15 | C1 | C2 |
|---|---|---|---|---|---|---|---|---|---|---|
| Amine | EDA | DETA | XA70 | ECA29 | N4 | TAN | MXDA | MXDA | EDA | DETA |
| Gel Time | 60 | 43 | 57 | 48 | 47 | 96 | 87 | 69 | 65 | 50 |
| Mins | | | | | | | | | | |

Clear Coat Properties @ 25° C.

| | | | | | | | | | | |
|---|---|---|---|---|---|---|---|---|---|---|
| BK-TFST | 7:15 | 2;45 | 3:15 | 2:30 | 2:45 | 3:15 | 4:00 | 4:00 | 4;45 | 5:15 |
| Phase II/III [h] | 10:45 | 3:30 | 5:30 | 3:45 | 3:45 | 4:15 | 5:00 | 5:15 | 13:00 | 7:00 |
| Appearance | Clear | Clear | Haze | Clear | Haze | Clear | Clear | Clear | Clear | Clear |
| | | | oily | oily | | | | | | |
| Perzoz | 234 | 267 | 252 | 275 | 278 | 219 | 284 | 308 | 244 | 215 |
| Hardness [7 d] | | | | | | | | | | |
| Conical Bend | 2% | 2% | 2% | 2% | 2% | 2% | 2% | 2% | 2% | 2% |
| Elongation | | | | | | | | | | |
| Water spot | F/G | F/G | P/G | F/G | F/G | F/G | G/G | G/G | F/G | F/G |
| resistance | | | | | | | | | | |
| [1 d/7 d] | | | | | | | | | | |

Clear Coat Properties @ 5° C.

| | | | | | | | | | | |
|---|---|---|---|---|---|---|---|---|---|---|
| BK-TFST | 22:30 | 7:45 | 10:00 | 8:45 | 10:15 | 10:00 | 12:00 | 16:15 | 11:00 | 10:00 |
| Phase II/III [h] | >24:00 | 12:00 | 15:30 | 14:00 | 14:30 | 12:30 | 14:30 | 21:30 | 24:00 | 27:00 |
| Appearance | Haze | Haze | Haze | Haze | Haze | Slight | Clear | Clear | Haze | Haze |
| | oily | | tacky | tacky | oily | Haze | | | oily | oily |
| Perzoz | 72 | — | 70 | 68 | 175 | 198 | 197 | 227 | 79 | — |
| Hardness [7 d] | | | | | | | | | | |
| Water spot | P/F | P/F | P/F | P/F | P/F | P/F | P/F | P/F | P/F | P/F |
| resistance | | | | | | | | | | |
| [1 d/7 d] | | | | | | | | | | |
| Carbamation | P/F | F/F | P/F | P/F | P/G | P/F | P/G | P/G | P/F | P/F |
| resistance | | | | | | | | | | |
| [1 d/7 d] | | | | | | | | | | |

As illustrated by Table 3, the clear coatings of the examples studied, vary in performance depending upon the nature of the (poly)amine used. The major benefits of the phenalkamine curing agents produced via the cardanol-DMA exchange method as defined in the invention result in coating systems, which demonstrate faster dry speed development when cured at low temperatures (5° C.). This is illustrated with the DETA formulation as defined by Example 5 versus comparative example C2 and the MXDA formulations as defined by Example 12 versus Example 15. In general, the phenalkamines produced via the exchange process, give good mechanical and barrier properties, which are typical of products of this class. In the case of Example 3, the added benefit of faster dry speed isn't observed due to the low inherent active N—H of EDA and low overall functionality.

### Novel Phenalkamine Curing Agent—Polyamide Co-Curing Agent Composition

Performance properties obtained for clear coatings formulated by blending examples from the invention with a polyamide curing agent are illustrated in Table 4. The polyamide used in this study is Ancamide® 350A, available from Evonik Corp.

TABLE 4
Coating Dry Speed Properties of Novel Phenalkamine-Polyamide Blends

| Formulation | Ex 16 | Ex 17 | Ex 18 |
|---|---|---|---|
| Ancamide 350A | 100 | 50 | 50 |
| Example 8 | | 50 | |
| Example 11 | | | 50 |
| Curing agent viscosity | 14,000 | 2,760 | 4,400 |
| @25° C. [mPa · s] | | | |
| BK-TFST @25° C. | 5:45 | 4:30 | 3:30 |
| Phase II/III [h] | 18:00 | 7:15 | 4:30 |
| Appearance after | Clear | Clear | Clear |
| 24 hrs @25° C. | Glossy | Glossy | Glossy |
| BK-TFST @5° C. | 44:00 | 19:00 | 15:00 |
| Phase II [h] | | | |
| Appearance after | Clear | Clear | Clear |
| 48 hrs @5° C. | Tacky | Glossy | Glossy |

As highlighted in Table 4, the phenalkamines from this invention are readily compatible with an industry standard high solids polyamide hardener. The result of which is a curing agent composition with a significantly lower handling viscosity, and a composition which exhibits a faster development of thin film cure speed at both 25° C. and 5° C. The addition of the phenalkamines from Examples 8 & 11 to the polyamide, also provides films with high gloss and free from tack after 24 hrs cure.

1. A method for producing a phenalkamine comprising the steps of
a. preparing a Mannich base by reacting cardanol with a secondary amine of formula 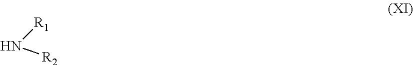
with R₁ and R₂ being, independently of each other, a C₁-C₆ alkyl or aryl group, and an aldehyde, and
b. reacting the Mannich base with a compound with at least one alkylene or aralkylene group and at least two amino groups.

2. A method according to claim 1,
wherein the secondary amine is dimethylamine.

3. A method according to claim 1,
wherein the compound with at least one alkylene or aralkylene group is represented by the following formula (VIII)
NH₂[(CH₂)ₓNH]ₘ—Z (VIII)
wherein x=2-10, m=1-10, Z is H or (CH₂)ₚ(OH) and p is 2 or 3.

4. A method according to claim 1,
wherein the compound has the following formula (IX)

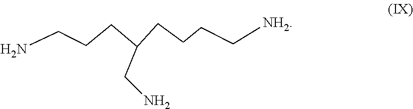 [SMILES: NCCCCC(CN)CCCN]

5. A method according to claim 1,
wherein the compound has the following formula (X)

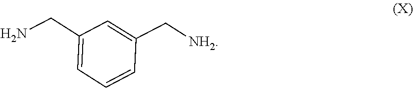

6. A method according to claim 3,
wherein the compound of formula (VIII) is a mixture of triethylenetetramine, tetraethylenepentamine, hydroxyethyl diethylenetriamine and hydroxyethyl triethylenetetramine.

7. A method according to claim 3,
wherein in formula (VIII) Z═H, m=5-10 and x=2.

8. A method according to claim 3,
wherein the compound of formula (VIII) is N,N′-1,2-ethanediyl-bis-1,3-propanediamine.

9. A phenalkamine produced by a process according to claim 1.

10. (canceled)

11. (canceled)

12. A phenalkamine of formula (III) 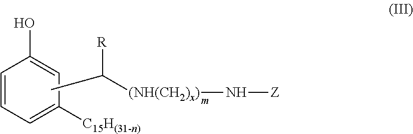
with n=0, 2, 4 or 6, Z═H, m=5-10, x=2 and R═H, a C₁-C₆ alkyl or Ph.

13. A phenalkamine of formula (VI) 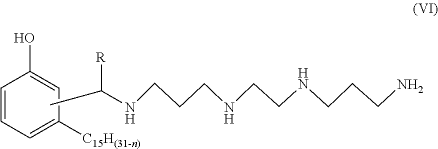
with n=0, 2, 4 or 6, and R═H, a C₁-C₆ alkyl or Ph.

14. An amine-epoxy composition comprising the reaction product of a phenalkamine according to claim 9 and an epoxy component.

15. An article of manufacture comprising the amine-epoxy composition according to claim 14.

16. The article of manufacture of claim 15, wherein the article is an adhesive, a coating, a primer, a sealant, a curing compound, a construction product, a flooring product, a composite product, a laminate, a potting compound, a grout, a filler, a cementitious grout, or self-leveling flooring.
